(19) United States
(12) Patent Application Publication
(10) Pub. No.: US 2019/0175031 A1
(43) Pub. Date: Jun. 13, 2019

(54) HAND-BASED BLOOD PRESSURE MEASUREMENT SYSTEM, APPARATUS AND METHOD

(71) Applicant: Indian Institute of Technology Bombay, Powai Mumbai (IN)

(72) Inventors: Santosh B. NORONHA, Powai Mumbai (IN); Sushanth D. POOJARY, Mumbai (IN); Pradip GATKINE, Nagpur (IN)

(21) Appl. No.: 16/321,975

(22) PCT Filed: Aug. 7, 2017
(86) PCT No.: PCT/IN2017/050325

§ 371 Date: Jan. 30, 2019

(30) Foreign Application Priority Data
Aug. 5, 2016 (IN) 201621026154

(51) Int. Cl. A61B 5/021; A61B 5/0402; A61B 5/00; A61B 5/0408; A61B 5/0472

(52) U.S. Cl. CPC A61B 5/02125; A61B 5/0402; A61B 5/6806; A61B 5/02427; A61B 5/0472; A61B 5/6898; A61B 5/04085

(57) ABSTRACT

The present invention discloses a hand based blood pressure measurement system, apparatus and method. In one embodiment two or more hand-based electrocardiogram (E.C.G.) sensor and one or more hand-based arterial pulse wave measurement sensor are used. These respectively determine the E.C.G. waveform associated with the heart beat blood transmission pulse and the GO arterial pulse/wave measurement. This data is used to measure the pulse transit time for one or more heart beats. In one embodiment the arterial pulse measurement is done using a photoplethysmogram sensor. In another embodiment the arterial pulse measurement is done using a pressure sensor.

### BACKGROUND

### A. Technical Field

The present invention relates to blood pressure measurement and monitoring. More particularly, the invention relates to non-invasive blood pressure measurement.

### B. Background of the Invention

Blood pressure assessment is an integral part of clinical practice. The two methods for blood pressure measurement are direct and indirect method. The direct method is the criterion standard and consists of using an intra-arterial catheter to obtain a measurement. This method, however due to its invasiveness and its inability to be applied to large groups of asymptomatic individuals for hypertension screening is not practically used.

The indirect method comprises of using a non-invasive method wherein conventional mercury sphygmomanometers and many related blood pressure measurement devices work on the principle of occluding blood supply in the brachial artery. The existing solutions make use of a cuff that needs to be inflated and then deflated. The said conventional process takes 3-4 minutes to complete. Such occlusion, if retained for a long time can be dangerous and can lead to severe and irreversible damage to the arteries in older patients. Thus limiting the utility of conventional sphygmomanometers for continuous monitoring.

The disadvantage of using cuff-based blood pressure monitoring device, is an extensive amount of time is spent to apply the cuff, and in its inflation and deflation. The traditional blood pressure monitoring devices are bulky and a mercury spill may occur that can be hazardous. Further the blood pressure monitors must be kept upright on a flat surface during measurement; the gauge must be read at eye level for accuracy. Further it may not work well for the hearing or visually impaired or for those unable to perform the hand movement needed to squeeze the bulb and inflate the cuff.

Further automated oscillometric devices available, remove the errors that can occur with manual measurements but are not without faults. The inaccuracy and inconsistency of the oscillometric devices has some concern such as using these devices in certain populations, such as hypotensive, hypertensive, trauma, or cardiac arrhythmia patients, can lead to inappropriate management. Further, the time spent in inflation and deflation of the cuffs continues to be a problem.

Therefore, there is a need for a cuff-less blood pressure monitoring device that is safe and accurate, screens large number of patients in various scenarios for instance, rural health camps and public hospitals, where a reliable measurement and recording of blood pressure is required to be done in the shortest possible time.

### SUMMARY OF THE INVENTION

The present invention discloses a hand based blood pressure measurement system, apparatus and method. In one embodiment two or more hand-based electrocardiogram (E.C.G.) sensor and one ore more, hand-based arterial pulse wave measurement sensor are used. These respectively determine the E.C.G. waveform associated with the heart beat blood transmission pulse and the arterial pulse/wave measurement. This data is used to measure the pulse transit time for one or more heart beats.

In one embodiment the arterial pulse measurement is done using a photoplethysmogram sensor. In another embodiment the arterial pulse measurement is done using a pressure sensor.

The system uses a combination of pulse waveforms and E.C.G. waveform, as recorded simultaneously using one or more hands of a subject. In one exemplary embodiment the blood pressure measurement may be done by placing the hands on an hand placement surface adjustable hand-print template that has sensors mounted on them. The said surface may also have adjustable hand print template thereon for providing guidance to user on placement of hand. In another exemplary embodiment a glove based implementation may be available where the sensors are integrated on the inner side of the glove. In yet another embodiment the sensors may be placed on the mobile phone cover surface.

The data obtained from the various sensors are used to identify includes E.C.G. critical points from E.C.G. waveform. The E.C.G. critical points may include R-peak and, Q and S foot points. Further, arterial pulse waveform critical points from arterial pulse waveform. The arterial pulse waveform critical points may include pulse peak point, pulse foot-point, maximum-slope point, dicrotic notch point.

The above calculated E.C.G. critical points and arterial pulse waveform critical points are used to compute the pulse transit time. Further, using said user parameter data and the pulse transit time to compute blood pressure. Various mathematical models may be chosen based on user parameters such as age, gender, height. For example, a physics-based transformation function may be used based on the user parameters to extract the systolic and diastolic blood pressure from the pulse transit time.

Further, blood pressure calculated may be compared with AI Classifier to decide on normalization. The normalized blood pressure may thus be obtained.

This approach gives beat-to-beat blood pressure and can be used for continuous blood pressure monitoring scenarios as well. This method also gives blood pressure as a function of time within a pulse/beat waveform. This measurement of peripheral blood pressure as a function of time is important to extrapolate central aortic blood pressure waveform.

### BRIEF DESCRIPTION OF THE DRAWINGS

Reference will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 illustrates a schematic diagram of a hand based blood pressure measurement system as per an embodiment herein.

FIG. 2 shows a hand based blood pressure apparatus as per an embodiment herein in a hand-placement-surface implementation.

FIG. 3 shows another embodiment of the hand based blood pressure apparatus in a mobile-phone-cover-surface implementation.

FIG. 4 shows another embodiment of the hand based blood pressure apparatus in a hand-glove implementation.

FIG. 5 shows a flow diagram illustrating the step by step process of capturing and measuring the blood pressure level as per an embodiment herein.

### DETAILED DESCRIPTION OF THE INVENTION

The present invention discloses a hand-based blood pressure measurement system, apparatus and method. In one embodiment two or more hand-based electrocardiogram (E.C.G.) sensor and one ore more, hand-based arterial pulse wave measurement sensor are used. These respectively determine the E.C.G. waveform associated with the heart beat and the arterial pulse wave measurement. This data is used to measure the pulse transit time for one or more heart beats.

In one embodiment the arterial pulse measurement is done using a photoplethysmogram sensor. In another embodiment the arterial pulse measurement is done using a pressure sensor.

In the following description, for purpose of explanation, specific details are set forth in order to provide an understanding of the invention. It will be apparent, however, to one skilled in the art that the invention may be practiced without these details. One skilled in the art will recognize that embodiments of the present invention, some of which are described below, may be incorporated into a number of different blood pressure measurement systems, methods and devices. The embodiments of the present invention may be present in hardware, software or firmware. Structures and devices shown below in block diagram are illustrative of exemplary embodiments of the invention and are meant to avoid obscuring the invention. Furthermore, connections between components within the figures are not intended to be limited to direct connections. Rather, data between these components may be modified, re-formatted or otherwise changed by intermediary components.

Reference in the specification to “one embodiment” or “an embodiment” means that a particular feature, structure, characteristic, or function described in connection with the embodiment is included in at least one embodiment of the invention. The appearances of the phrase “in one embodiment” in various places in the specification are not necessarily all referring to the same embodiment. The headings and subheadings used in the document are not intended to limit the content therein to respective heading or subheading. Rather, they are used to help reader navigate and prevent obscuring the invention.

### a. Overview

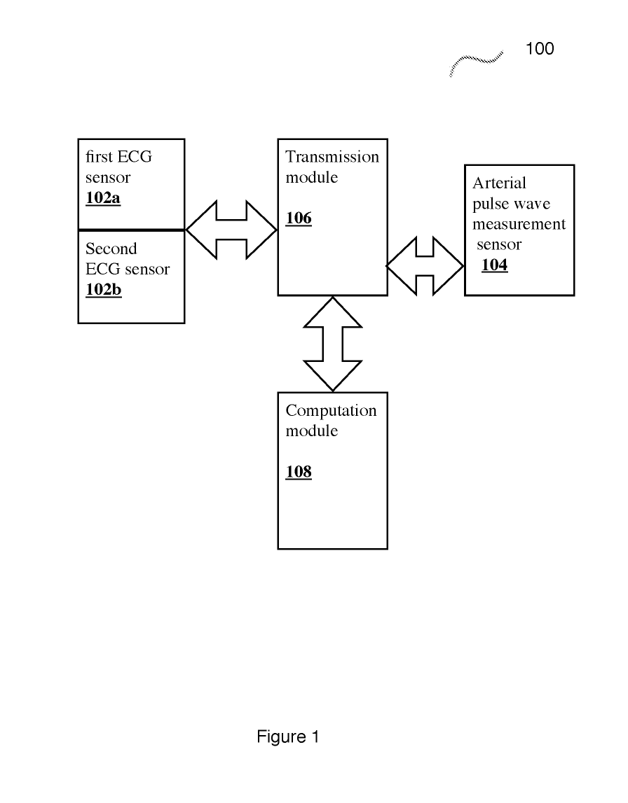

FIG. 1 shows an embodiment of a hand-based blood pressure measurement system as per an embodiment herein. The system may use a combination of pulse waveforms and E.C.G. waveform, as recorded simultaneously using one or more hands of a subject. In one exemplary embodiment the blood pressure measurement may be done by placing the hands on a hand placement surface that has sensors mounted on them. The said surface may also have adjustable hand print template thereon for providing guidance to user on placement of hand. In another exemplary embodiment a glove based implementation may be available where the sensors are integrated on the inner side of the glove. In yet another embodiment the sensors may be placed on the mobile phone cover surface.

Once the hands are placed on the hand-print template, the E.C.G. and arterial pulse measurement can be completed that gives us pulse transit time. The pulse transit time may be used to calculate the blood pressure.

Further the above described technology can be extended to possible hardware implementation embodiment such as gearable, wearable blood pressure measuring device that can be held, stuck or patched on hands or shoulders of individual.

The blood pressure measurement system 100 may comprise of two or more hand-based E.C.G. sensors 102a and 102b and one or more arterial pulse wave measurement sensor 104. The arterial pulse wave measurement sensor may be based on optical principle such as for example photoplethysmogram sensor. The pulse wave measurement sensor may be based on pressure measurement, such as for example piezoelectric pressure sensor.

For the measurement of E.C.G. at least two E.C.G. sensors are required. Further, for the arterial pulse wave measurement at least one arterial pulse wave measurement sensor is needed. More than two E.C.G. sensors improve the signal to noise ratio and also make the system more robust to loss of contact at a contact point on the hand skin. Further, the first E.C.G. sensor may be present on one hand and the second E.C.G sensor may be present on another hand. Both the first and the second E.C.G. sensors may also be present on same hand. Further, the arterial pulse wave measurement sensor may be present on any hand. If more than one arterial pulse wave measurement sensor is used the other arterial pulse wave measurement sensor may be placed on same hand as the first arterial pulse wave measurement or on a different hand.

The E.C.G. sensor used may be coated with tin to prevent oxidation of the typical copper surface sensors. This improves the accuracy of the data acquired due to reduced noise, which was earlier caused due to the amorphous oxides preventing seamless contact of the sensor surface and the portion of the skin of the hand at a contact point. Furthermore, graphene coated sensors may be used. These graphene coated sensors may be mounted on a surface in the required formation in which they are to contact the hand skin. These sensors are so mounted with a view to reduce signal artifact due to muscle tremors and jitter in the hands. Graphene known for its electron mobility, high surface area, and electrical conductivity enables high accuracy measurements to be taken.

A data transmission module 106 may be present which receives raw data from the said sensors and processes it for transmission to the computation module 108. The computing module may be wirelessly connected to the data transmission module. A wired version may also exist. These two modules may be present physically attached or may be present as separate units that are in wired or wireless communication with each other.

The transmission module 106 may be configured to receive raw analog signal from each of the sensors, and add timestamps to each. Further, it may be configured to digitize the signal/s. Further, the transmission module may be configured to encrypt the resulting digital signal with timestamp resulting in digital data that may be transmitted to computation module. The transmission module may also be configured to reduce noise using noise filtering and motion artefact correction. The motion artefact correction may include correcting for breathing artefacts, baseline wander and natural movement of hand. The noise filtering may include filtering the high-frequency noise from the electronic components and filtering the frequencies of power-grid, such as 50 Hz frequency in India. The transmission may occur using wired or wireless transmission. For example, wired, bluetooth or wifi based transmission may take place.

The transmission module thus transmits one or more transmission signal indicative of the first E.C.G. signal, the second E.C.G. signal and the first arterial pulse wave signal. There might be scenario where a transmission signal is sent for each E.C.G. signal separately. In another scenario these may be combined to form E.C.G. waveform before transmission. Also, noise reduction and motion artefact may be reduced before transmitting the transmission signal.

The computation module 108 may be configured to receive digital data and decrypt the same. The computation module may be configured to identify systolic and diastolic pressure points along with critical points such as R-peak in E.C.G., pulse peak point, pulse foot-point, maximum-slope point in the pulse and dicrotic notch point in the pulse and arterial pulse wave critical points such as for example . . . . Using the systolic and diastolic pressure points, critical points and personal information of individual the computation module may calculate pulse transit time. The computation module may also be configured to measure average pulse transit time values or average blood pressure values.

### b. Hand-Placement Based Implementation

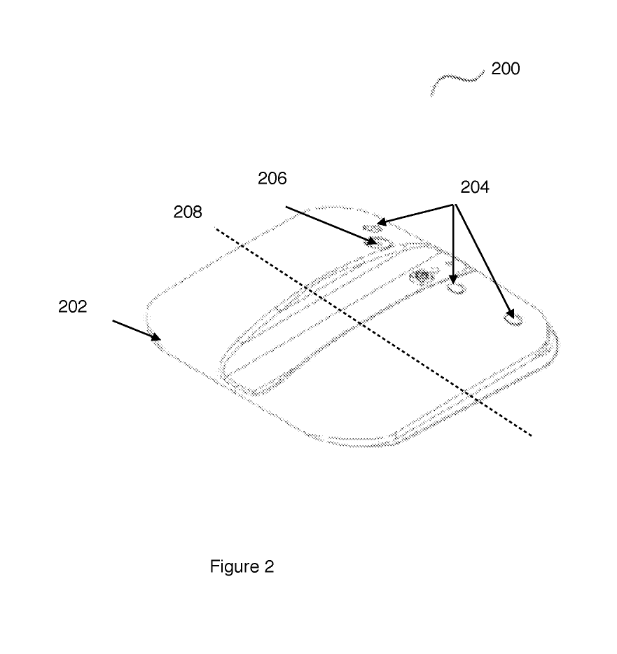

FIG. 2 shows a hand based blood pressure apparatus as per an embodiment herein. The apparatus comprises of a hand-resting surface 202 over which a subject may place hands. An E.C.G. sensor 204 may be present at two or more points on the said surface. In the exemplary embodiment shown in FIG. 2, the E.C.G. sensors are present at a location on the surface where the right hand's index and ring fingers are likely to rest. Further, in the shown exemplary embodiment an E.C.G. sensor is also present at a location on the surface where the left middle finger is likely to rest. In this example a first E.C.G. sensor is touching a first contact point on the first hand that happens to be the index finger of the right hand and a second E.C.G. sensor is touching a second contact point on the second hand which happens to be ring finger of the right hand. Further in this example, an extra E.C.G. sensor is present touching the middle finger of the left hand. The various contact points needed to be in contact with the various sensors may be of different sizes. This may depends on the sensitivity and other qualitative factors of the sensors and type of user. In exemplary embodiment they are around 0.1 centimeter square of lesser. In another exemplary embodiment they may be 5 centimeter square of more as well. Different shapes depending on the kind of contact the skin of the user is likely to have may be possible, such as for example, circular, square, hexagonal, oval and other suitable shapes.

An arterial pulse wave measurement sensor 206 may be present at one or more points on the said surface. In the exemplary embodiment shown in FIG. 2 the arterial pulse wave measurement sensors are present at a location on the surface where the left hand's index finger is likely to rest.

The surface 202 may have the cutouts to allow for portion of hand to be in contact with the sensors which are placed along the surface 202, or underneath it. In another embodiment, the cutouts 212 and the sensors 204 and 206, may be present at various points on the said surface. For example, the E.C.G. sensor may be present at or near a point where the thumb meets the palm. In this example the contact point for that sensor is on the palm. Also, the arterial pulse wave measurement sensor 206 may be present in contact with other fingers. Various sensors may also be present on only one side of the surface 202 for example, the part where the left hand rests or the part where the right hand rests.

Further, the surface 202 may be curved outward along an axis 208 allowing better ease of placement of hand and contact of various sensors with the hand.

In another embodiment, the surface 202 may comprise of an adjustable hand-print template that allows positioning of the hands suitably. The adjustable hand-print template may be made from anti-microbial plastic. The hand-print templates may be provided accommodating decrease or increase the size of the hand print template depending on the need of the individual, at least one support may be provided on the lower side of the hand-print template for ease of placing the hand and proper contact with the hand-print template. The hand-print template may be present in form of depression on the surface 202. Further, a transmission module, may be present beneath the surface 202.

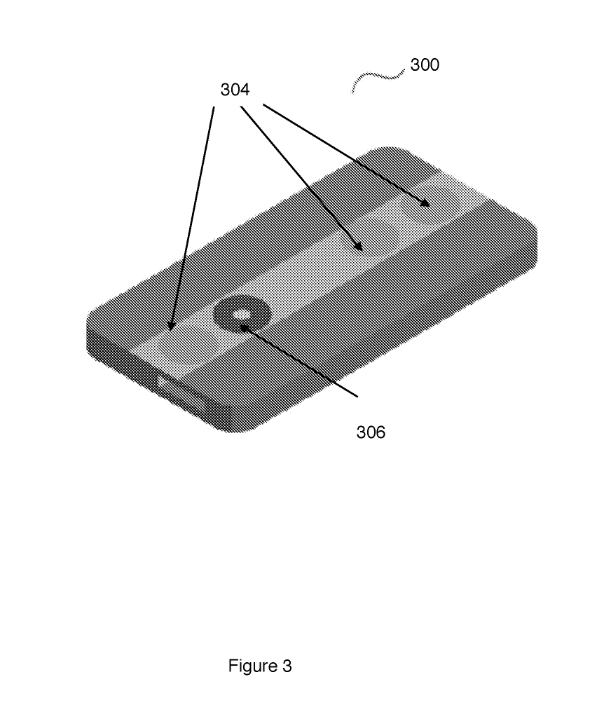

The another exemplary embodiment the surface 202 may be embodied into a mobile phone cover as shown in FIG. 3. The surface may allow for portion of hand to be rested thereon such that the contact points of the hand are in contact with the various sensors such as E.C.G. sensors 304 (three in number) and arterial pulse wave sensor 306.

This exemplary embodiment allows quick and accurate blood pressure measurement with high throughput and ease of use. This may be used in a health ATM setup where, such a device may be installed at a kiosk allowing users to measure their blood pressure with little or no assistance.

The said apparatus acts as a blood pressure monitoring device and as heart rate and pulse rate monitoring device. The apparatus can also be used to give E.C.G. measurements.

### c. Glove Based Implementation

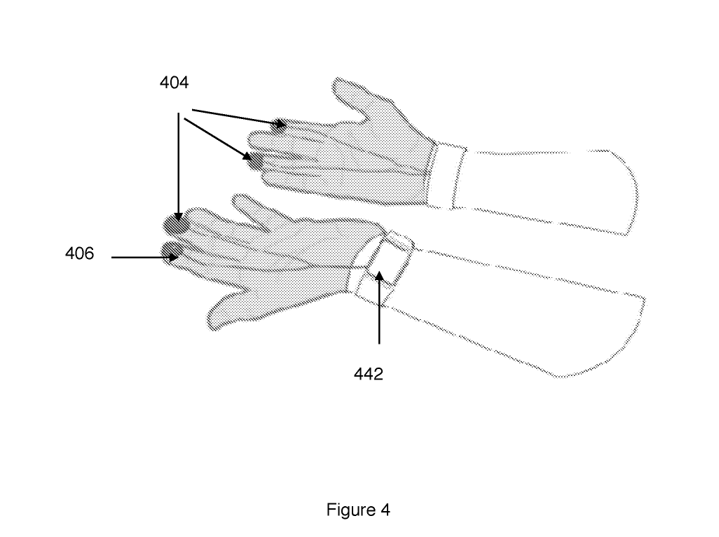

FIG. 4 shows another embodiment of the hand based blood pressure apparatus. In this example the hand based blood pressure measurement apparatus is embodied in a glove. The glove may be made from various materials suitable for the purpose, such as for example fabric, plastic, synthetic fibres.

An E.C.G. sensor 404 may be present at two or more points in contact with the hand when the glove is worn by the user. In the exemplary embodiment shown in FIG. 4 an E.C.G. sensor is present at a location on the surface where the right hand's index finger and another E.C.G. sensor where ring finger is likely to rest. Further, in the shown exemplary embodiment, an E.C.G. sensor is also present at a location on the surface where the left middle finger is likely to rest. A conductive fabric based sensor may be used for E.C.G. measurement allowing better flexibility and maneuverability.

An arterial pulse wave measurement sensor 406 may be present at one or more points of the glove allowing contact of the pulse wave measurement sensor with the hand at desired contact point. In the exemplary embodiment shown in FIG. 4 the arterial pulse wave measurement sensor is present at a location on the surface where the left hand's index finger is likely to be present when the glove is worn. The glove 402 may have the cutouts in case the said sensors are present on the outer surface of the glove.

In another embodiment, the cutouts and the sensors 404 and 406, may be present at other points on the said surface. For example, an E.C.G. sensor may be present at or near a point where the thumb meets the palm. Also, the arterial pulse wave measurement sensor 406 may be present on other fingers. Various sensors may also be present on only one side of the glove pair 402, for example, on the left glove or the right glove.

Further, a data transmission module 442 may be present along the wrist portion of one or both glove pair, which receives raw data from the said sensors and processes it for transmission to a computation device such as for example a mobile phone, a laptop, a tablet or a standalone computing module. The standalone computing module may also be attached physically to the glove.

The glove implementation is particularly useful for long term repeated measurements. Also, in patients experiencing anxiety over blood pressure measurement such implementation would provide more accurate results. This embodiment may also be suitable for ambulatory blood pressure measurement.

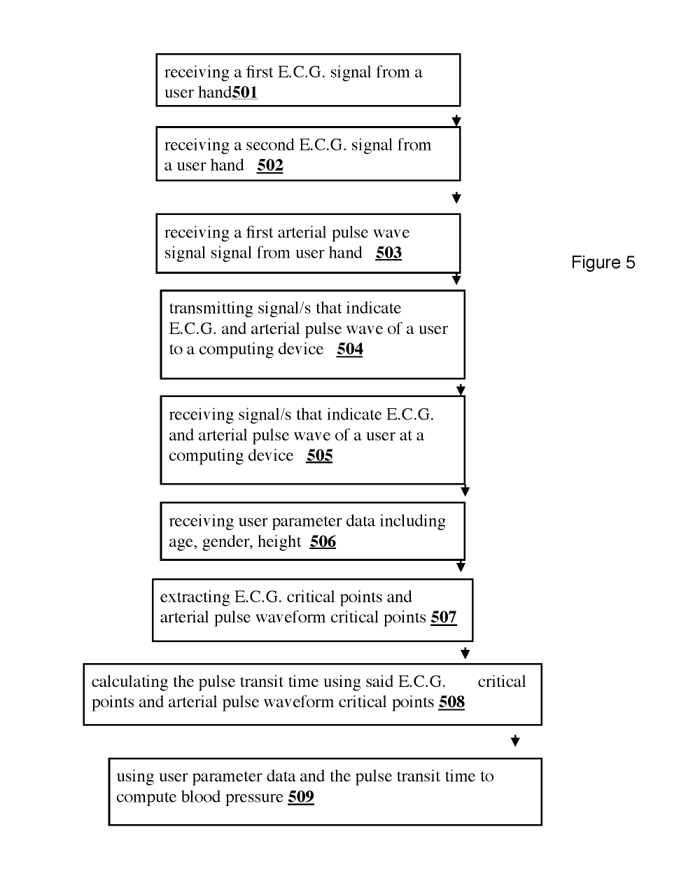

FIG. 5 shows a method for measuring blood pressure as per an embodiment herein.

Firstly a user is requested to allow his/her hand's contact points to get in contact with various sensors. The user may rest one or more hand facing flattened palms down on a surface, such that at least a portion of the skin of the hand touches the first E.C.G. sensor on a first contact point, the second E.C.G. sensor on a second contact point and a first arterial wave pulse measurement sensor on a third contact point. The contact points may exist on various parts of the skin of the user. For example, In the example as shown in FIG. 2, a first E.C.G. sensor is touching a first contact point on the first hand that happens to be the index finger of the right hand and a second E.C.G. sensor is touching a second contact point on the second hand which happens to be ring finger of the right hand. Further in this example, an extra E.C.G. sensor is present touching the middle finger of the left hand. Furthermore, an arterial pulse wave measurement sensor may be present at one or more points. In the exemplary embodiment shown in FIG. 2 the arterial pulse wave measurement sensor is present at a location on the surface where the left hand's index finger is likely to rest. Here the third contact point is thus the left hand's index finger.

In another embodiment the user starts by inserting one or both hands inside one or more glove/s, such that at least a portion of the skin of the hand touches the first E.C.G. sensor on a first contact point, the second E.C.G. sensor on a second contact point and the first arterial wave pulse measurement sensor on a third contact point. In a single hand operation the left or the right hand can be inserted into a single hand glove that contains at least two E.C.G. sensor and at least one arterial pulse wave measurement sensor. In two hands operation a hand glove is available for each glove and the contact points are spread over both hands.

Further, the method comprises receiving 501 a first E.C.G. signal from a first E.C.G. sensor in contact with the skin of a first hand of a user on a first contact point and receiving 502 a second E.C.G. signal from a first E.C.G. sensor in contact with the skin of a second hand of a user on a second contact point. Further, a first arterial pulse wave signal is received 503 from a first sensor for arterial wave pulse measurement in contact with the skin of the first hand or the second hand of a user on a third contact point.

Further step includes transmitting 504 one or more transmission signal indicative of the first E.C.G. signal, the second E.C.G. signal and the first arterial pulse wave signal to a computation module for measurement of blood pressure. There might be scenario where a transmission signal is sent for each E.C.G. signal separately. In another scenario these may be combined to form a simultaneous E.C.G. waveform and an arterial pulse waveform before transmission. Also, noise reduction and motion artefact may be reduced before transmitting the transmission signal.

Further step includes receiving 505 one or more transmission signal indicative of, the first E.C.G. signal, the second E.C.G. signal and the first arterial pulse wave signal. As described above this may include signal related to individual sensor or a simultaneous E.C.G. waveform and an arterial pulse waveform. This may be received in wired or wireless manner at a computation device. The computing device may also receive 506 user parameter data such as for example age, gender and height. The further step includes extracting 507 E.C.G. critical points from E.C.G. waveform constructed from the one or more transmission signal. The E.C.G. critical points may include R-peak and, Q and S foot points. Further, the computation module also extracts 507 arterial pulse waveform critical points from arterial pulse waveform constructed from one or more transmission signal. The arterial pulse waveform critical points may include pulse peak point, pulse foot-point, maximum-slope point, dicrotic notch point

The above calculated E.C.G. critical points and arterial pulse waveform critical points are used to compute 508 the pulse transit time. Further, using said user parameter data and the pulse transit time to compute 509 blood pressure. Various mathematical models may be chosen based on user parameters such as age, gender, height. For example, a physics-based transformation function may be used based on the user parameters to extract the systolic and diastolic blood pressure from the pulse transit time.

Further, blood pressure calculated may be compared with an artificial intelligence classifier, derived using previous empirical data, to decide on normalization. The blood pressure value is then displayed and the action thereafter is terminated, thus completing the process of measuring the blood pressure.

This approach gives beat-to-beat blood pressure and can be used for continuous blood pressure monitoring scenarios as well. This method also gives blood pressure as a function of time within a pulse/beat waveform. This measurement of peripheral blood pressure as a function of time is important to extrapolate central aortic blood pressure waveform.

Further the above described method can be extended to possible embodiment such as gear able, wearable blood pressure measuring device that can be held, stuck or patched on hands or shoulders.

The present invention converts the pulse wave velocity to blood pressure. The pulse wave velocity is measured using the time-delay between the hand-based E.C.G. signal measured using at least two E.C.G. sensors and an arterial pulse wave signal measured using a pulse plethysmogram (PPG) or pulse pressure sensor. Additionally, the user parameters such as user's age, gender and height are also used to determine the pulse wave velocity from pulse transit time as well as to determine transformation function from pulse wave velocity to blood pressure. In other words, the present invention converts the pulse transit time—measured using hand-based ECG and hand-based arterial pulse wave signal—into blood pressure by using additional user parameters such as age, gender and height.

A person skilled in the art may realize that the location of various sensors used may be extended slightly further from the hands without deviating from the basic principles of the invention. Such example include having sensors placed on wrist or surrounding area.

The present invention measures blood pressure reliably within a few seconds, thus speeding up the process and minimizing the discomfort for the patient. Further, stiffening of arteries is a dominant precursor of most of the cardiovascular diseases. Pulse wave transit time is a direct indicator of arterial stiffness. Thus, the added information of pulse wave transit time is of great clinical importance in cardiovascular diagnostics. This added information can help doctors make a more informed decision about the medications and further treatment to monitor the effect of medications over a period of time.

The present invention requires very little to no occlusion of the arteries and is thus well-suited for both short-term and long term continuous monitoring of blood pressure. Further this approach also provides beat-to-beat blood pressure values, thus helping to identify some particular cardiovascular diseases which result in large blood pressure variability from beat to beat. Further the cost of this device is comparable to that of conventional sphygmomanometers and is much cheaper than any of the devices measuring pulse wave transit time.

The present invention can also be commercially applied in hospital setups, as personalized device, ambulatory measurement and in psychological test and criminal interrogations to monitor the response of blood pressure and pulse wave transit time for different kinds of stimuli/questions. Further, it finds application in demanding scenarios such as rural health camps and public hospitals where a reliable measurement and recording of blood pressure is required to be done in the shortest possible time.

Although the present invention has been described in detail with reference to certain preferred embodiments thereof, other versions are possible.

One skilled in the art will realize the disclosure may be embodied in other specific forms without departing from the disclosure or essential characteristics thereof. Various presently unforeseen or unanticipated alternatives, modifications, variations or improvements therein may be subsequently made by those skilled in the art, each of which is also intended to be encompassed by the disclosed embodiments and within the scope of appended claims.

This disclosure is not limited to the particular, devices and methods described, as these may vary. The terminology used in the description is for the purpose of describing the particular versions or embodiments only, and is not intended to limit the scope.

We claim:

1. An apparatus for blood pressure measurement comprising:
a first electrocardiogram sensor configured to contact the skin of a first hand on a first contact point and produce a first electrocardiogram signal;
a second electrocardiogram sensor configured to contact the skin of a second hand on a second contact point and produce a second electrocardiogram signal;
a first sensor for arterial wave pulse measurement configured to contact the skin of the first hand or the second hand on a third contact point and produce a first arterial pulse wave signal;
a transmission module configured to transmit one or more transmission signal indicative of the first electrocardiogram signal, the second electrocardiogram signal and the first arterial pulse wave signal.

2. The apparatus for blood pressure measurement as in claim 1, wherein:
the first hand is same as the second hand.

3. The apparatus for blood pressure measurement as in claim 1, wherein:
the transmission module is further configured to perform one or more of digitizing, timestamping, encryption and noise reduction of: the first electrocardiogram signal, the second electrocardiogram signal and the first arterial pulse wave signal, to produce the one or more transmission signals.

4. The apparatus for blood pressure measurement as in claim 1, wherein:
the first sensor for arterial wave measurement is a photoplethysmogram sensor.

5. The apparatus for blood pressure measurement as in claim 1, wherein:
the first sensor for arterial wave measurement is a pressure sensor.

6. The apparatus for blood pressure measurement as in claim 1, wherein the first electrocardiogram sensor and the second electrocardiogram sensor is tin coated.

7. The apparatus for blood pressure measurement as in claim 1, further comprising a computation device configured to:
receive the one or more transmission signal indicative of the first electrocardiogram signal, the second electrocardiogram signal and the first arterial pulse wave signal;
receive user parameter data;
extract electrocardiogram critical points from an electrocardiogram waveform constructed from the one or more transmission signal;
extract arterial pulse waveform critical points from an arterial pulse waveform constructed from the one or more transmission signal;
measure the pulse transit time using said electrocardiogram critical points and arterial pulse waveform critical points;
using said user parameter data and the pulse transit time to compute blood pressure.

8. The apparatus for blood pressure measurement as in claim 1, further comprising:
a hand glove configured to allow inserting a hand therein;
the first electrocardiogram sensor positioned along the material of the glove such that the first contact point touches said first electrocardiogram sensor when a user's hand is inserted in the hand glove;
the second electrocardiogram sensor positioned along the material of the glove such that the second contact point touches said second electrocardiogram sensor when a user's hand is inserted in the hand glove;
the first arterial wave pulse measurement sensor positioned along the material of the glove such that the third contact point touches said first arterial wave pulse measurement sensor when a user's hand is inserted in the hand glove.

9. The apparatus for blood pressure measurement as in claim 1, wherein:
the first contact point is the tip of the face of an index finger of the left or the right hand, the second contact point is the tip of the face of a ring finger of the left or the right hand, and the third contact point is the tip of the face of a middle finger of the left or the right hand.

10. The apparatus for blood pressure measurement as in claim 1, further comprising:
a surface for resting one or more hand facing flattened palms down, such that at least a portion of the skin of the hand touches said surface;
the first electrocardiogram sensor positioned along the surface such that the first contact point touches said first electrocardiogram sensor;
the second electrocardiogram sensor positioned along the surface such that the second contact point touches said second electrocardiogram sensor;
the first arterial wave pulse measurement sensor positioned along the surface such that the third contact point touches said first arterial wave pulse measurement sensor.

11. The apparatus for blood pressure measurement as in claim 10, wherein the surface is a surface on a mobile phone cover.

12. A method for blood pressure measurement comprising the steps of:
receiving a first electrocardiogram signal from a first electrocardiogram sensor in contact with the skin of a first hand of a user on a first contact point;
receiving a second electrocardiogram signal from a second electrocardiogram sensor in contact with the skin of a second hand of a user on a second contact point;
receiving a first arterial pulse wave signal signal from a first a first sensor for arterial wave pulse measurement in contact with the skin of the first hand or the second hand of a user on a third contact point;
transmitting one or more transmission signal indicative of the first electrocardiogram signal, the second electrocardiogram signal and the first arterial pulse wave signal to a computation module for measurement of blood pressure.

13. The method for blood pressure measurement as in claim 12, wherein:
the first hand is same as the second hand.

14. The method for blood pressure measurement as in claim 12, further comprising the steps of:
receiving the one or more transmission signal indicative of the first electrocardiogram signal, the second electrocardiogram signal and the first arterial pulse wave signal;
receiving user parameter data;
extracting electrocardiogram critical points from an electrocardiogram waveform constructed from the one or more transmission signal;
extracting arterial pulse waveform critical points from an arterial pulse waveform constructed from one or more transmission signal;
calculating the pulse transit time using said electrocardiogram critical points and arterial pulse waveform critical points;
using said user parameter data and the pulse transit time to compute blood pressure.

15. The method for blood pressure measurement as in claim 12, comprising the step of:
resting one or more hand facing flattened palms down on a surface, such that at least a portion of the skin of the hand touches the first electrocardiogram sensor on a first contact point, the second electrocardiogram sensor on a second contact point and the first arterial wave pulse measurement sensor on a third contact point.

16. The method for blood pressure measurement as in claim 12, comprising the step of:
inserting one or more hand inside a glove, such that at least a portion of the skin of the hand touches the first electrocardiogram sensor on a first contact point, the second electrocardiogram sensor on a second contact point and the first arterial wave pulse measurement sensor on a third contact point.

17. The method for blood pressure measurement as in claim 14, wherein:
the user parameter data include age, gender and height.

18. The method for blood pressure measurement as in claim 14, wherein:
electrocardiogram critical points include R-peak, Q and S foot points.

19. The method for blood pressure measurement as in claim 14, wherein:
arterial pulse waveform critical points include pulse peak point, pulse foot-point, maximum-slope point, dicrotic notch point.

20. The method for blood pressure measurement as in claim 15, wherein:
the surface is a surface on a mobile phone cover.

21. A non-transitory computer readable medium storing a blood pressure measurement program that, when executed by a processor, causes a computer to perform the following blood pressure measurement method:
receiving one or more transmission signal indicative of a first electrocardiogram signal, a second electrocardiogram signal and a first arterial pulse wave signal obtained from contact points on the skin of one or more hand of a user;
receiving user parameter data including age, gender, height;
extracting electrocardiogram critical points from electrocardiogram waveform constructed from the one or more transmission signal;
extracting arterial pulse waveform critical points from arterial pulse waveform constructed from one or more transmission signal;
calculating the pulse transit time using said electrocardiogram critical points and arterial pulse waveform critical points;
using said user parameter data and the pulse transit time to compute blood pressure.
